(19) United States
(12) Patent Application Publication
(10) Pub. No.: US 2004/0127404 A1
(43) Pub. Date: Jul. 1, 2004

(54) Methods for treating autoimmune diseases

(75) Inventors: Nam H. Dang, Houston, TX (US); Timothy Enns, Pleasanton, CA (US)

Correspondence Address: WILSON SONSINI GOODRICH & ROSATI, 650 PAGE MILL ROAD, PALO ALTO, CA, 943041050

(73) Assignees: Board of Regents, The University of Texas System, Austin, TX (US); Supergen, Inc., San Ramon, CA (US)

(21) Appl. No.: 10/742,492

(22) Filed: Dec. 18, 2003

Related U.S. Application Data
(63) Continuation of application No. 09/620,254, filed on Jul. 20, 2000, now abandoned.
(60) Provisional application No. 60/145,365, filed on Jul. 22, 1999.

(51) Int. Cl. A61K 38/13; A61K 31/7076; A61K 31/66; A61K 31/525

(52) U.S. Cl. 514/11; 514/251; 514/109; 514/400; 514/43; 514/171

(57) ABSTRACT

The present invention provides methods for treating a patient with an autoimmune disease by administering an inhibitor of adenosine deaminase (ADA) such as pentostatin and its analogs and derivatives.

### CROSS-REFERENCE TO RELATED APPLICATIONS

This application is a continuation application of Ser. No. 09/620,254, filed Jul. 20, 2000, which is a continuation of Provisional application Serial No. 60/145,365, filed Jul. 22, 1999, both of which are incorporated herein by reference in their entirety.

### BACKGROUND OF THE INVENTION

1. Field of the Invention

This invention relates to treatment of autoimmune diseases and more specifically to the administration of inhibitors of adenosine deaminase (ADA) such as pentostatin and analogs and derivatives thereof.

2. Description of Related Art

A wide range of degenerative diseases, the so-called autoimmune diseases, are caused by the immune system attacking a person's own cells. Autoimmune diseases are characterized by such immuno-dysfunction that circulating autoantibodies or cells are generated directing against normal autologous tissues.

Initiation of autoimmune diseases is not well understood, but involves both genetic predisposition and environmental factors. Such diseases are usually classified clinically in a variety of ways. In light of affected parts by the diseases, there are, for example, degenerative diseases of supporting tissues and connective tissues; autoimmune degenerative diseases of salivary glands, particularly Sjogren's disease; autoimmune degenerative diseases of kidneys, particularly systemic lupus erythematodes (SLE) and glomerulonephritis; autoimmune degenerative diseases of joints, particularly rheumatoid arthritis; and autoimmune degenerative diseases of blood vessels such as generalized necrotizing angitis and granulomatous angitis; and multiple sclerosis. Alternatively, autoimmune diseases can be classified in one of the two different categories: cell-mediated disease (i.e. T-cell) or antibody mediated disorders. Examples of cell-mediated autoimmune diseases include multiple sclerosis, rheumatoid arthritis, autoimmune thyroiditis, and diabetes mellitus. Antibody-mediated autoimmune disorders include myasthenia gravis and SLE.

The current treatments for both categories of autoimmune diseases involve administration of drugs such as methotrexate, cyclophosphamide, azathioprine and cyclosporin A. Steroid compounds such as prednisone and methylprednisilone are also employed in regimen.

Other strategies have been developed to target autoantibody. For example, antigen-based heteropolymers have been used to specifically target both complementary receptor site (CR1) on a primate erythrocyte and a target pathogenic autoantibody (U.S. Pat. No. 5,879,679). In the heteropolymer, a monoclonal antibody specific for binding to a CR1 site on a primate erythrocyte is cross linked to an antigen specific for a target pathogenic autoantibody.

There still exists the need for improved treatment for autoimmune diseases which acts on the immune system and which has a function mechanism different from those currently available.

### SUMMARY OF THE INVENTION

The present invention provides novel methods for treating autoimmune diseases by exploiting the signal transduction pathways of T-cell activation via ADA and CD26. Applicants believe that by inhibiting ADA- and CD26-mediated T lymphocyte activation, inflammatory responses of the body and symptoms of the autoimmune disease should be reduced.

A method is provided which comprises administrating to a patient suffering from an autoimmune disease in need thereof a therapeutically effective amount of an anti-ADA agent. The anti-ADA agent may either act as an ADA enzyme inhibitor to prevent the enzyme from deaminating adenosine, or bind to ADA to prevent ADA from binding to the T-cell surface CD26.

According to this embodiment, the anti-ADA agent is may be an analog or derivative of adenosine which can bind to ADA but cannot be deaminated. Examples of such adenosine analogs and derivatives include, but are not limited to, pentostatin (2′-deoxycoformycin), fludarabine monophosphate, 2-chloro-2′-deoxyadenosine, 2′-deoxyadenosine, 3′-deoxyadenosine and dideoxyadenosine.

The anti-ADA agents can be administrated into a patient with an autoimmune disease in many pharmaceutically-acceptable ways. The anti-ADA agent can be administrated intravenously, intramuscularly, orally, by inhalation, parenterally, intraperitoneally, intraarterially, transdermally, sublingually, nasally, through use of suppositories, transbuccally, liposomally, adiposally, intraocularly, subcutaneously, intraarticularly, intrathecally, topically, or through local administration.

In one variation of the embodiment, the method comprises administrating pentostatin to the patient.

According to this variation, pentostatin is administrated orally by incorporating into an orally dosage form such as tablets and soft or hard gel capsules. For example, pentostatin may be administrated orally in the form of a tablet.

In another variation of the embodiment, pentostatin is administrated parenterally by dissolving in a pharmaceutically-acceptable solvent such as saline or other infusion fluids. For example, pentostatin may be dissolved in a solution comprising pentostatin.

The solution above may further include an excipient in an amount sufficient to enhance the stability of the solution. Examples of the excipient includes, but are not limited to, sorbitol, mannitol and cyclodextrin such as α-, β-, and γ-cyclodextrin and modified, amorphous cyclodextrin such as hydroxy-substituted α-, β- and γ-cyclodextrin.

The anti-ADA agents according to the present invention can be administered alone or in combination with each other. For example, pentostatin may be coadministered with deoxyadenosine to enhance inhibition of T lymphocyte. The anti-ADA agents can also be used in combination with other drugs known for the treatment of autoimmune diseases, such as cyclosporin A, methotrexate, cyclophosphamide, azathiprine and steroids. Alternatively, the anti-ADA agent can be coadministered with a H2 blocker such as cimetidine to enhance the bioavailability of the anti-ADA agent.

The method can be used to treat patients suffering from autoimmune diseases such as multiple sclerosis, Graves' disease, systemic lupus erythematosus, diabetes mellitus, aseptic meningitus, systemic scleroderma, adult-onset idiopathic hypoparathyroidism and membranous glomerulonephritis.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention provides novel methods for treating autoimmune diseases. In essence, the method comprises administrating to a patient suffering from the autoimmune disease a therapeutically effective amount of an anti-ADA agent such as an adenosine analog.

It is believed that the method operates by exploiting the signal transduction pathways of T-lymphocyte or T-cell activation via ADA and CD26. It is also believed that by inhibiting ADA- and CD26-mediated T lymphocyte activation, inflammatory responses of the body and symptoms of the autoimmune disease should be reduced.

1. Autoimmune Diseases

Autoimmune diseases refer to a wide range of degenerative diseases caused by the immune system attacking a person's own cells. Autoimmune diseases are usually classified clinically in a variety of ways. In light of affected parts by the diseases, there are, for example, degenerative diseases of supporting tissues and connective tissues; autoimmune degenerative diseases of salivary glands, particularly Sjogren's disease; autoimmune degenerative diseases of kidneys, particularly systemic lupus erythematodes (SLE) and glomerulonephritis; autoimmune degenerative diseases of joints, particularly rheumatoid arthritis; and autoimmune degenerative diseases of blood vessels such as generalized necrotizing angitis and granulomatous angitis; and multiple sclerosis. Alternatively, autoimmune diseases can be classified in one of the two different categories: cell-mediated disease (i.e. T-cell) or antibody mediated disorders. Examples of cell-mediated autoimmune diseases include multiple sclerosis, rheumatoid arthritis, autoimmune thyroiditis, and diabetes mellitus. Antibody-mediated autoimmune disorders include myasthenia gravis and SLE.

2. CD26 and ADA

CD26 is a 110-kD cell surface glycoprotein and widely distributed on epithelial cells of various tissues, including the liver, kidney and intestine. CD26 is generally expressed on the CD4 memory/helper population and also strongly expressed on subsets of both activated CD4 and CD8 T-cells (Fox, D. A. et al. (1984) J. Immunol. 133:1250-1256). This unique population of human CD4 cells is the only one that can respond to recall antigens, induce B-cell immunoglobulin (IgG) synthesis and activate MHC-restricted cytotoxic T cells (Dang, N. H. et al. (1990) J. Immunol. 144:4092-4100).

CD26 is ecto-enzyme that resides on the cell surface and has dipeptidyl peptidase IV (DPPIV) activity. This ecto-enzyme can cleave Xaa-Pro or Xaa-Ala dipeptides from the N-termini of polypeptides (Watanabe, Y. et al. (1993) Res. Commun. Chem. Pathol. Pharmacol. 81:323-330).

Recently CD26 has been found to be highly expressed on the surface of T-cells from patients suffering from autoimmune diseases such as multiple sclerosis, diabetes mellitus, rheumatoid arthritis, Graves' disease and systemic lupus erythematodes (SLE) (Constantinescu, C. S,. et al. (1995) J. Neurol. Sci. 130: 178-182; Mizokami A. et al. (1996) J. Rheumatol. 23:2022-2026; Golubovic E. et al. (1996) Srp. Arh. Celok. Lek. 124: Suppl. 1:43-44; Nishkawa, Y. et al. (1995) Rinsho Byori 43:1057-1060; Plana M. et al. (1994) Clin. Immunol. Immunopathol. 72: 227-232).

ADA is a 41 kD protein expressed in all tissues with highest expression in lymphocytes. ADA participates in the purine metabolism where it degrades either adenosine or 2′-deoxyadenosine producing inosine or 2′-deoxyinosine, respectively. It has been found that the activity of ADA is subject to changes depending upon the degree of activity of the cell, i.e. whether differentiation or proliferation occurs (Trotta, P. P. and Balis M. E. (1977) Cancer Research 37: 2297-2305).

3. Methods for Treating Autoimmune Diseases

In one embodiment, a method is provided which comprises administrating to a patient suffering from an autoimmune disease a therapeutically effective amount of an anti-ADA agent. The anti-ADA agent may either act as an ADA enzyme inhibitor to prevent the enzyme from deaminating adenosine, or binds to ADA to prevent ADA from binding to the T-cell surface CD26.

According to this embodiment, the anti-ADA agent is a structural analog of adenosine which binds to ADA but cannot be deaminated. Examples of such anti-ADA agent includes, but are not limited to, pentostatin (2′-deoxycoformycin or NIPENT®, fludarabine monophosphate, 2-chloro-2′-deoxyadenosine, 2′-deoxyadenosine, 3′-deoxyadenosine and dideoxyadenosine. Prodrugs of these anti-ADA agents such as phosphates, esters and amides may also be used in the practice of this invention. For example, prodrugs of pentostatin may be used to increase bioavailability through selective bioconversion.

In particular, pentostatin is a tight-binding inhibitor of ADA which has been used as a therapeutic agent for a number of disorders including hairy cell leukemia and acute lymphocytic leukemia. 2′-deoxycoformycin is available as NIPENT7 (Supergen, San Ramon, Calif.). Pentostatin is believed to act in vivo to increase adenosine concentration by inhibiting adenosine deaminase (ADA) through competitive binding. Such binding is biologically quite tight, with a binding coefficient of Kᵢ of 2.5×10⁻²M. Through this inhibition, pentostatin may slow in vivo degradation of adenosine and/or prevent ADA from binding to CD26, thus increasing the concentration and activity of adenosine near the cell surface of T cells.

Pentostatin has been used in the laboratory to mimic the effects of inherited adenosine deaminase deficiency which is the underlying biochemical defect in ⅓ to ½ of cases of non-X-linked severe combined immunodeficiency syndrome. Mitchell, B. et al., “Purinogenic immunodeficiency disease: Clinical features and molecular mechanisms”, Ann Int. Med. 92:826-831m (1980). The clinical use of pentostatin has primarily been with T lymphocyte malignancies including T-cell acute lymphocytic leukemia, lymphocytic lymphoma, chronic lymphocytic leukemia and mycosis fungoides (Sezary Syndrome).

The anti-ADA agent can be incorporated into a variety of formulations for the treatment of autoimmune diseases. More particularly, the anti-ADA agent can be formulated into pharmaceutical compositions by combination with appropriate, pharmaceutically acceptable carriers or diluents, and may be formulated into preparation in solid, semi-solid, or liquid forms, such as tablets, capsules, pills, powders, granules, dragees, gels, slurries, ointments, solutions, suppositories, injections, inhalants and aerosols.

The anti-ADA agent can be administrated into a patient with an autoimmune disease in many pharmaceutically-acceptable ways. The anti-ADA agent can be administrated intravenously, intramuscularly, orally, by inhalation, parenterally, intraperitoneally, intraarterially, transdernally, sublingually, nasally, through use of suppositories, transbuccally, liposomally, adiposally, intraocularly, subcutaneously, intraarticularly, intrathecally, topically, or through local administration.

The anti-ADA agents can be administered alone, in combination with each other, or they can be used in combination with other drugs known for the treatment of autoimmune diseases, such as cyclosporin A, methotrexate, cyclophosphamide, azathioprine and steroids (e.g. prednisone and methyprednisone).

For injection, the anti-ADA agents can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the anti-ADA agents may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the anti-ADA agents can be formulated readily by combining with pharmaceutically acceptable carriers that are well known in the art. Such carriers enable the compounds to be formulated as tablets, pills, dragees, capsules, emulsions, lipophilic and hydrophilic suspensions liquids, gels, syrups, slurries, suspensions and the like, for oral ingestion by a patient with an autoimmune disease. Pharmaceutical preparations for oral use can be obtained by mixing the anti-ADA agent with a solid excipient, optionally grindign a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylprrolidone (PVP). If desired, disintegrating agents may be added, such as the cross-linked polyvinyl pyrrolidone, agar, or alginic acid or a salt thereof such as sodium

In the oral dosage forms, it may be useful to include antioxidants or preservatives. Antioxidants that may be used are sodium sulphite, sodium hydrogen sulphite, sodium metabisulphite, ascorbic acid, ascorbylpalmitate, -myristate, -stearate, gallic acid, gallic acid alkyl ester, butylhydroxyamisol, nordihydroguaiaretic acid, tocopherols as well as synergists (substances which bind heavy metals through complex formation, for example lecithin, ascorbic acid, phosphoric acid ethylene diamine tetracetic acid, citrates, tartrates). Addition of synergists substantially increases the antioxygenic effect of the antioxidants.

Preservatives also be used in the oral dosage forms. Examples of preservatives include sorbic acid, p-hydroxybenzoic acid esters (for example lower alkyl esters), benzoic acid, sodium benzoate, trichloroisobutyl alcohol, phenol, cresol, benzethonium chloride, chlorhexidine and formalin derivatives.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active drug doses.

Pharmaceutically preparations of anti-ADA agents which can be used orally include push-fit capsules made of gelatin, as well as soft, sealed capsules made of gelatin and a plasticizer, such as glycerol or sorbitol. The push-fit capsules can contain the anti-ADA agent in admixture with filler such as lactose, binders such as starches, and/or lubricants such as talc or magnesium sterate and, optionally, stabilizers. In soft capsules, the anti-ADA agent may be dissolved or suspended in suitable liquids, such as fatty oils, liquid paraffin, or liquid polyethylene glycols. In addition, stabilizers may be added. All formulations for oral administration should be in dosage suitable for such administration.

For buccal administration, the anti-ADA agent may take the form of tablets or lozenges formulation in conventional manner.

For administration by inhalation, anti-ADA agents of the present invention are conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, for example, gelatin for use in an inhaler or insufflator may be formulated containing a powder mix of the compound and suitable powder base such as lactose or starch.

In another variation of the embodiment, the anti-ADA agent is administrated parenterally, e.g. by bolus injection or continuous infusion. Formulations of anti-ADA agent for injection may be presented in unit dosage form, such as in ampules or in multidose containers, with an added preservative. The formulations may take such forms as suspension, solutions or emulsion in oily or aqueous vehicles, and may contain formulatory agents such as suspending, stabilizing and/or dispersing agents.

Pharmaceutical formulations for anti-ADA agents for parenteral administration include aqueous solutions of the anti-ADA agent in water-soluble form. Additionally, suspensions of the anti-ADA agent may be prepared as appropriate oily injection suspension. Suitable lipophilic solvents or vehicles include fatty oils such as sesame oil, or synthetic fatty acid esters, such as ethyl oleate or triglycerides, or liposomes. Aqueous injection may contain substances which increase the viscosity of the suspension, such as sodium carboxymethyl cellulose, sorbitol, or dextran. Optionally, the suspension may also contain suitable solubilizers or agents which increase the solubility of the compounds to allow for the preparation of highly concentrated solutions. Examples of such solubilizers include, but are not limited to, cyclodextrin such as α-, β-, and γ-cyclodextrin and modified, amorphous cyclodextrin such as hydroxy-substituted α-, β- and γ-cyclodextrin. Alternatively, the active ingredient may be in powder form for constitution with a suitable vehicle, e.g., sterile pyrogen-free water, before use.

The anti-ADA agents may also be formulated in rectal compositions such as suppositories or retention enemas, e.g. containing conventional suppository bases such as cocoa butter, carbowaxes, polyethylene glycols or other glycerides, all of which melt at body temperature, yet are solidified at room temperature.

In addition, the anti-ADA agents may be formulated as a depot preparation for administration by implantation (e.g., subcutaneously or intramuscularly) or by intramuscular injection. Thus, for example, the anti-ADA agent may be formulated with suitable polymeric or hydrophobic materials (e.g., as an emulsion in an acceptable oil) or ion exchange resins, or as sparingly soluble derivative, for example, as sparingly soluble salt.

Alternatively, anti-ADA agents can be administrated to a patient with an autoimmune disease by employing other delivering systems such as liposome-mediated drug delivery. Liposomes and emulsions are well known examples of delivery vehicles or carriers for hydrophobic drugs. Certain organic solvents such as dimethylsulfoxide also may be employed, although usually at the cost of greater toxicity. Additionally, the anti-ADA agents can be delivered using a sustained-release system, such as semipermeable matrices of solid hydrophobic polymers containing the anti-ADA agent. Various types of sustained-release materials have been established and are well known by those skilled in the art. Sustained-release capsules may, depending on their chemical nature, release the anti-ADA agent for a few weeks up to over 100 days.

In addition, the anti-ADA agents may be administrated in a targeted drug delivery system, for example, in a liposome coated with a cell-specific antibody. Such liposomes will be targeted to and taken up selectively by the cell of interest (a specific subset of T cells). Liposomes and emulsions are well known examples of delivery vehicles or carriers for hydrophobic drugs. For example, long-circulating, i.e., stealth, liposomes can be employed. Such liposomes are generally described in U.S. Pat. No. 5,013,556, the teaching of which are hereby incorporated by reference.

Alternatively, the anti-ADA agents may also be administrated with various agents to reduce acid concentration in the stomach. This reduces acid lability and allows for enhanced concentrations of anti-ADA agents for enhanced gastric and/or intestinal absorption. For example, the anti-ADA agent may be coadministered with a H2 inhibitor such as cimetidine, an acid neutralizer.

The method of the present invention includes administrating to a subject in need thereof (e.g a patient or animal with an autoimmune disease) an anti-ADA agent composition wherein the anti-ADA agent is contained in a therapeutically effective amount. The amount of composition administered will, of course, dependent on the subject being treated, on the subject's weight, the severity of the affliction, the manner of administration and the judgment of the prescribing physician. Determination of an effective amount is well within the capability of those skilled in the art, especially in the light of the detailed disclosure provided herein.

In a preferred embodiment, the method comprises administrating pentostatin to the patient.

According to this embodiment, pentostatin may be orally administrated to a patient with an autoimmune in need thereof in the form of a tablet. The patient may also be pretreated with an H2 blocker such as cimetidine to enhance the bioavailability of pentostatin.

Alternatively, pentostatin may be parenterally administrated to a patient carry an autoimmune disease. For example, pentostatin may be administered intravenously in 50 cc. 0.9 Normal Saline (NS) over a 30 minute period, together with intravenous hydration of 500 cc of 0.9 NS over 1 hour before and after pentostatin. In a preferable embodiment, a pentostatin containing solution includes pentostatin in an amount of about 0.02 to about 1 weight percent, more preferably about 0.1 to about 0.3 weight percent, where the weight is based on the total compositional weight. Furthermore, the solution may contain additional ingredients, such as conventional pharmaceutical excipients. In a preferred embodiment, the pentostatin solution additionally comprises mannitol, and sterile water. Sodium hydroxide or hydrochloric acid may also be added to adjust pH.

Pentostatin as an inhibitor of adenosine deaminase may be synergized in inhibiting T lymphocytes by the administration of deoxyadenosine. In another preferred embodiment, pentostatin and may be coadministered with deoxyadenosine.

The method can be used to treat patients suffering from autoimmune diseases. The method is preferred to be used to treat those patients diagnosed with an autoimmune disease and tested having elevated levels of the T cell activation marker CD26. Patients include, but are not limited to those with multiple sclerosis, Graves' disease, systemic lupus erythematosus, diabetes mellitus, aseptic meningitus, systemic scleroderma, adult-onset idiopathic hypoparathyroidism and membranous glomerulonephritis.

For example, an adult patient who suffers from systemic lupus erythematosus and is HIV negative may be treated with an ADA inhibitor such as pentostatin. Pentostatin (e.g., NIPENT® supplied by SuperGen, Inc, Dublin, Calif.) may be administered to the patient intravenously at 1-20 mg/m², preferably at 2-10 mg/m², and more preferably at 4-6 mg/m². The dosing schedule may be once a month or more frequent depending on the prognosis of the patient.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

What is claimed is:

1. A method for treating an autoimmune disease selected from the group consisting of multiple sclerosis, Graves' disease, systemic lupus erythematosus, diabetes mellitus, aseptic meningitis, systemic scleroderma, adult onset idiopathic hypoparathyroidism, and membranous glomerulonephritis, comprising:
testing a group of patients having said autoimmune disease for elevated levels of expressed T-cell surface CD26 relative to the levels of expressed T-cell surface CD26 of a normal person;
selecting from the group one or more patients having elevated levels of T-cell surface CD26; and
then administrating to the selected patients a therapeutically effective amount of pentostatin.

2. The method according to claim 1, wherein pentostatin is administrated intravenously, intramuscularly, orally, by inhalation, parenterally, intraperitoneally, intraarterially, transdermally, sublingually, nasally, through use of suppositories, transbuccally, liposomally, adiposally, intraocularly, subcutaneously, intraarticularly, intrathecally, topically, or through local administration.

3. The method according to claim 1, further comprising administering to the patient an anti-autoimmune disease drug selected from the group consisting of cyclosporin A, methotrexate, cyclophosphamide, azathioprine and steroids.

4. The method according to claim 1, further comprising administering to the patient an anti-acid drug.

5. The method according to claim 4, wherein the anti-acid drug is cimetidine.

6. The method according to claim 1, wherein pentostatin is administrated orally.

7. The method according to claim 6, wherein pentostatin is administered in an oral dosage form selected from the group consisting of tablet, pill, dragee, capsule, emulsion, lipophilic and hydrophilic suspension, gel, syrup, and slurry.

8. The method according to claim 1, wherein pentostatin is administrated parenterally.

9. The method according to claim 8, wherein pentostatin is present in a solution comprising pentostatin.

10. The method according to claim 9, wherein the pentostatin is present in a solution that additionally comprises mannitol and sterile water.
